(19) United States
(12) Patent Application Publication
(10) Pub. No.: US 2010/0056784 A1
(43) Pub. Date: Mar. 4, 2010

(54) PROCESS FOR THE PREPARATION OF VORICONAZOLE

(75) Inventors: Ernesto Duran Lopez, Barcelona (ES); Marcal Carreras i Vilagran, Tarragona (ES); Juan Contreras Lascorz, Girona (ES)

Correspondence Address: FROMMER LAWRENCE & HAUG, 745 FIFTH AVENUE- 10TH FL., NEW YORK, NY, 10151, US

(73) Assignee: MEDICHEM, S.A., GIRONA (ES)

(21) Appl. No.: 12/373,587

(22) PCT Filed: Jul. 12, 2007
(86) PCT No.: PCT/IB07/04408

§ 371 Date: Oct. 1, 2009

Related U.S. Application Data
(60) Provisional application No. 60/807,275, filed on Jul. 13, 2006.

(51) Int. Cl. C07D 401/06

(52) U.S. Cl. 544/333

(57) ABSTRACT

The present invention relates to an improved process for the preparation of Voriconazole.

### REFERENCE TO RELATED APPLICATIONS/INCORPORATION BY REFERENCE

Any foregoing applications, and all documents cited therein or during their prosecution (“application cited documents”) and all documents cited or referenced in the application cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

Citation or identification of any document in this application is not an admission that such document is available as prior art to the present invention.

### FIELD OF THE INVENTION

The present invention relates to an improved process for the preparation of Voriconazole.

### BACKGROUND OF THE INVENTION

Voriconazole is a commercially marketed pharmaceutically active substance known to be useful for the treatment of some fungal infections. Voriconazole has an empirical formula of C₁₆H₁₄F₃N₅O and a molecular weight of 349.3. Voriconazole is the international common accepted name for (2R,3S)-2-(2,4-difluorophenyl)-3-(5-fluoropyrimidin-4-yl)-1-(1H-1,2,4-triazol-1-yl)butan-2-ol, which is represented in formula (I).

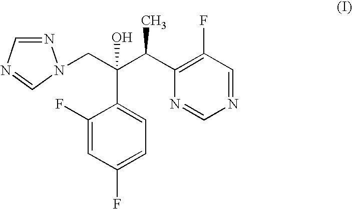

Voriconazole is a triazole antifungal agent. Voriconazole works principally by inhibition of cytochrome P450 14a-demethylase (P45014DM). This enzyme is in the sterol biosynthesis pathway that leads from lanosterol to ergosterol. Compared to fluconazole, voriconazole inhibits P45014DM to a greater extent. This inhibition is dose-dependent. Voriconazole is active following both oral and intravenous administrations. Oral (200 mg twice daily) and intravenous (3 to 6 mg/kg every 12 h) doses of Voriconazole have produced favorable response. Voriconazole is marketed under the name VFEND®. The VFEND® products are available as an I.V. solution, a powder for oral suspension (and hence an oral suspension), and film coated tablets for oral administration. VFEND® is for the treatment of some fungal infections. VFEND® is said to help fight life-threatening fingal infections, such as fungal infections in people who have a weak immune system, e.g., patients with cancer or patients who have received an organ or bone marrow transplant. VFEND® is said to have been proven effective against a type of fungus called Aspergillus. The following U.S. Patents are listed in the U.S. FDA's Orange Book as to VFEND®: U.S. Pat. No. 5,116,844; U.S. Pat. No. 5,134,127; U.S. Pat. No. 5,364,938; U.S. Pat. No. 5,376,645; U.S. Pat. No. 5,567,817; U.S. Pat. No. 5,773,443; and U.S. Pat. No. 6,632,803. Formulations, doses and uses of Voriconazole as available commercially in the VFEND® product, and as in these herein cited US patents may be employed in the practice of the herein invention.

All of the processes described in the literature for the preparation of Voriconazole involves the resolution of racemic Voriconazole (compound II) with (1R)-(−)-10-camphorsulfonic acid [(−)-CSA] to give (2R,3S)-2-(2,4-difluorophenyl)-3-(5-fluoropyrimidin-4-yl)-1-(1H-1,2,4-triazol-1-yl)-butan-2-ol (1R)-(−)-10-camphorsulfonate, that is, Voriconazole (1R)-(−)-10-camphorsulfonate (compound III). Voriconazole (compound I) is isolated from Voriconazole (1R)-(−)-10-camphorsulfonate (compound III) as illustrated in Scheme 1, below.

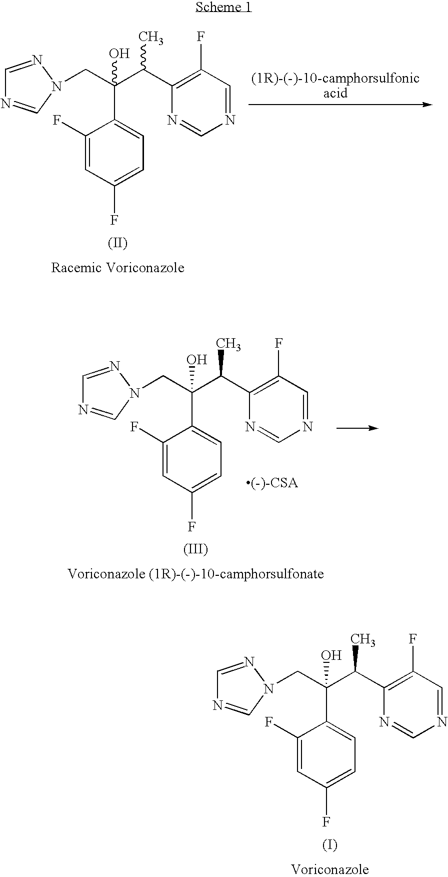

U.S. Pat. No. 5,567,817 describes the resolution of racemic Voriconazole (compound II) with (1R)-(−)-10-camphorsulfonic acid [(−)-CSA] in methanol (38 volumes) to give Voriconazole (1R)-(−)-10-camphorsulfonate (compound III) which is treated with dichloromethane and saturated aqueous sodium bicarbonate to give Voriconazole (compound I).

U.S. Pat. No. 6,586,594 describes the resolution of racemic Voriconazole (compound II) with (1R)-(−)-10-camphorsulfonic acid [(−)-CSA] in a mixture (30 volumes) of acetone (22.5 volumes)/methanol (7.5 volumes) or in acetone (approx. 10 volumes) followed by a treatment in a mixture of methanol and acetone. Voriconazole (compound I) is isolated from Voriconazole (1R)-(−)-10-camphorsulfonate (compound III) using dichloromethane and 40% aqueous sodium hydroxide solution, evaporation of the organic extract and crystallization with isopropanol.

However, each of these processes are extremely time consuming and consume vast quantities of solvent rendering them unsuitable for industrial application. Each set of resolution steps (volume of solvent) creates the potential for product loss. In addition, each of the processes in U.S. Pat. No. 5,567,817 and U.S. Pat. No. 6,586,594 require the use of a halogenated solvent (e.g. dichloromethane (methylene chloride)) to achieve the conversion of Voriconazole (1R)-(−)-10-camphorsulfonate (compound III) to Voriconazole (compound I). Use of these solvents creates undesirable environmental and regulatory concerns (e.g. dichloromethane is a known toxic agent and irritant; chloroform is a known carcinogenic agent, etc.)

Therefore, there still exists a need in the art for an efficient process for the resolution of racemic Voriconazole which maximizes enantiomeric and chemical purity of Voriconazole ((2R,3S)-2-(2,4-difluorophenyl)-3-(5-fluoropyrimidin-4-yl)-1-(1H-1,2,4-triazol-1-yl)-butan-2-ol) and minimizes or eliminates the presence of other enantiomers of Voriconazole (e.g., the (2S,3R)-enantiomer).

Citation or identification of any document in this application is not an admission that such document is available as prior art to the present invention.

### SUMMARY OF THE INVENTION

Surprisingly, the problems associated with isolating Voriconazole from racemic Voriconazole can be overcome by practicing the process of the invention.

The present invention provides an improved process for preparing Voriconazole (compound I) and its intermediates which comprises treatment of racemic Voriconazole (compound II) with (1S)-(+)-10-camphorsulfonic acid [(+)-CSA] and isolating the (2S,3R)-enantiomer of Voriconazole (1S)-(+)-10-camphorsulfonate (compound IV):

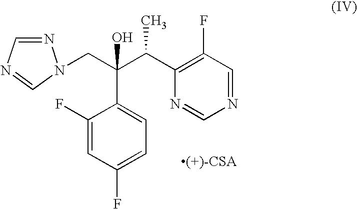

The remaining mother liquor contains an enriched mixture of Voriconazole (1S)-(+)-camphorsulfonate (compound V):

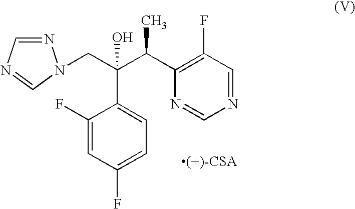

which is then converted into the free base form and subsequently treated with (1R)-(−)-10-camphorsulfonic acid to form Voriconazole (1R)-(−)-10-camphorsulfonate (compound III). The Voriconazole (1R)-(−)-10-camphorsulfonate (compound III) is treated with an alkaline solution to form Voriconazole (compound I) and is optionally separated via crystallization.

The present invention also provides a process which allows the preparation of Voriconazole (compound I) in high yield, high enantiomeric purity and high chemical purity which is achieved with lower amounts of solvent and without the use of halogenated solvents.

As used throughout the specification and claims:
(i) the term “Voriconazole” refers to the (2R,3S) enantiomer, i.e. (2R,3S)-2-(2,4-difluorophenyl)-3-(5-fluoropyrimidin-4-yl)-1-(1H-1,2,4-triazol-1-yl)butan-2-ol;
(ii) enantiomers of Voriconazole other than (2R,3S) will be referred to by the appropriate prefix, e.g. (2S,3R) Voriconazole refers to (2S,3R)-2-(2,4-difluorophenyl)-3-(5-fluoropyrimidin-4-yl)-1-(1H-1,2,4-triazol-1-yl)butan-2-ol;
(iii) the phrase “racemic Voriconazole” indicates the presence of Voriconazole, i.e. the (2R,3S) enantiomer, and also other enantiomers of Voriconazole;
(iv) the phrase “enriched with Voriconazole (1S)-(+)-10-camphorsulfonate” indicates that more Voriconazole (1S)-(+)-10-camphorsulfonate by weight is present than (2S,3R) Voriconazole (1S)-(+)-10-camphorsulfonate (in other embodiments within the scope of this definition, the ratio of Voriconazole (1S)-(+)-10-camphorsulfonate: (2S,3R) Voriconazole (1S)-(+)-10-camphorsulfonate is selected from the ranges consisting of about 60: about 40 to about 90: about 10; about 70: about 30 to about 80: about 20; and about 75: about 25—all ranges are by weight);
(v) the phrase “enriched with Voriconazole” indicates that more Voriconazole by weight is present than (2S,3R) Voriconazole (in other embodiments within the scope of this definition, the ratio of Voriconazole: (2S,3R) Voriconazole is selected from the ranges consisting of about 60: about 40 to about 90: about 10; about 70: about 30 to about 80: about 20; and about 75: about 25—all ranges are by weight);

Accordingly, it is an object of the invention to not encompass within the invention any previously known product, process of making the product or method of using the product such that applicant(s) reserve the right and hereby disclose a disclaimer of any previously known processes.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are apparent from and encompassed by, the following Detailed Description.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention. In the drawings:

FIG. 1: illustrates the Infrared (IR) spectrum of Voriconazole obtained in example 4

FIG. 2: illustrates the X-ray powder diffractogram (XRD) of Voriconazole obtained in example 4.

### DETAILED DESCRIPTION

Reference will now be made in detail to various embodiments of the invention. This invention may, however, be embodied in many different forms and should not be construed as limited to the embodiments set forth herein.

The present invention relates to an improved process for the preparation of Voriconazole (Compound I)

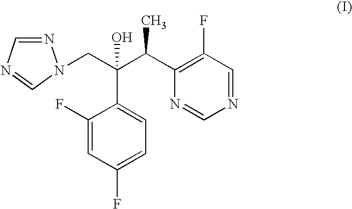

which comprises:
a) treating racemic Voriconazole (compound II) with (1S)-(+)-10-camphorsulfonic acid in an organic solvent to form a suspension containing (2S,3R)-enantiomer of Voriconazole (1S)-(+)-10-camphorsulfonate (compound IV);

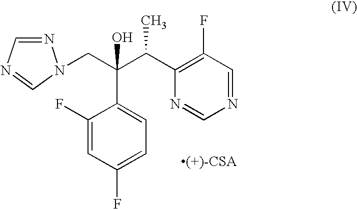

b) filtering the suspension and separating out the solid (2S,3R)-enantiomer of Voriconazole (1S)-(+)-10-camphorsulfonate (compound (IV) to form a mother liquor which is enriched with Voriconazole (1S)-(+)-10-camphorsulfonate (compound V);

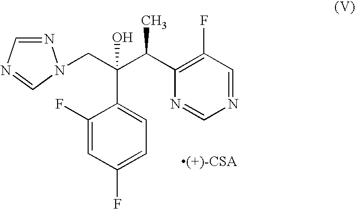

c) treating the mother liquor with an organic solvent and an aqueous alkaline solution to obtain an enriched mixture of Voriconazole (compound I);
d) treating the enriched mixture of Voriconazole with (1R)-(−)-10-camphorsulfonic acid in an organic solvent to obtain Voriconazole (1R)-(−)-10-camphorsulfonate (compound III);

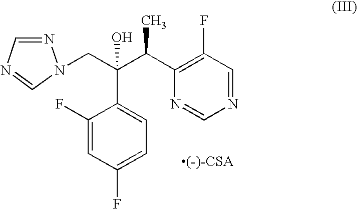

e) treating Voriconazole (1R)-(−)-10-camphorsulfonate (compound III) with an organic solvent and an aqueous alkaline solution to obtain Voriconazole (compound I); and
f) optionally purifying the Voriconazole;

Suitable bases and aqueous alkaline solutions which are compatible with the process include various inorganic bases. Such bases include, for example hydroxides of alkali metals or hydroxides of alkaline earth metals, carbonates or bicarbonates of alkali metals or carbonates or bicarbonates of alkaline earth metals. One embodiment of the aqueous alkaline solution is a saturated salt solution. In another embodiment of the aqueous alkaline solution, the solution is a saturated sodium bicarbonate solution.

Suitable organic solvents which are compatible with the process of the invention include but are not limited alcohols, esters, ketones, ethers, nitrites, hydrocarbons and mixtures thereof. In one embodiment of the invention, the solvents are selected from C₁-C₄ alcohols, methyl acetate, ethyl acetate and mixtures thereof. In another embodiment of the invention, the solvents are methanol, isopropanol, ethyl acetate and mixtures thereof.

The obtained Voriconazole (compound I) is characterized by having a high enantiomeric purity and a high chemical purity. In one embodiment of this invention, the enantiomeric purity is at least about 97.00% and the chemical purity is at least about 99.50%. In another embodiment of this invention, the enantiomeric purity is between about 97.50% to about 100.00% and chemical purity is between about 99.50% to about 100.00%. Surprisingly, these levels of purity are achieved within one process cycle, where a process cycle consists of performing steps a)-f) only once.

The invention is further described by the following non-limiting examples which further illustrate the invention, and are not intended, nor should they be interpreted to, limit the scope of the invention.

### Examples

### General Experimental Conditions:

i. Infrared Spectra

Fourier transform infrared spectra were acquired on a Perkin-Elmer 1600 series FTIR spectrometer and polymorphs were characterized in potassium bromide pellets.

ii. HPLC Method

HPLC, method A:

The chromatographic separation was carried out in a Daicel CHIRALCEL OD-H, 5 μm, 4.6 mm×250 mm column.

The mobile phase was prepared by mixing 850 ml of hexane with 150 ml of ethanol.

The chromatograph was equipped with a 254 nm detector and the flow rate was 1.0 ml/min at 20-25° C. Tests samples (10 μl) were prepared by dissolving 25 mg of sample in 25 ml of mobile phase.

HPLC, method B:

The chromatographic separation was carried out in a Symmetry C18, 3.5 μm, 4.6 mm×100 mm column.

The mobile phase A was a 0.010 M ammonium formate buffer, pH 4.0, which was prepared from 0.63 g of HCOONH₄ dissolved in 1000 ml of water. The pH was adjusted to 4.0 with formic acid. The mobile phase was mixed and filtered through a 0.22 μm nylon membrane under vacuum.

The mobile phase B was acetonitrile.

The chromatograph was programmed as follows:

Initial 0-8 min. 70% mobile phase A, 8-20 min. linear gradient to 20% mobile phase A, 20-40 min. isocratic 20% mobile phase A, 40-45 min. linear gradient to 70% mobile phase A and 45-55 min. equilibration with 70% mobile phase A.

The chromatograph was equipped with a 254 nm detector and the flow rate was 1.0 ml per minute at 20-25° C. Test samples (20 μl) were prepared by dissolving 50 mg of sample in 25 ml of acetonitrile.

HPLC, method C:

The chromatographic separation was carried out in a Kromasil 100Si, 5 μm, 4.6 mm×250 mm column.

The mobile phase was prepared by mixing 850 ml of hexane with 150 ml of ethanol.

The chromatograph was equipped with a 254 nm detector and the flow rate was 1.0 ml/min at 20-25° C. Tests samples (20 μl) were prepared by dissolving 25 mg of sample in 25 ml of mobile phase.

iii. Assay

350 mg of the sample was accurately weighed, dissolved in 70 ml of glacial acetic acid and titrated with 0.1 N HClO₄ VS determining the end point potentiometrically fitting the increases of volume to 0.05 ml in the proximities of equivalence point. Each ml of 0.1 N HClO₄ VS is equivalent to 34.93 mg of Voriconazole.

iv. Particle Size Distribution

Particle size measurement was obtained using a Malvern particle size analyser equipped with a 2 milliwatt Helium/Neon laser and a Fourier Transform lens system. The sample was run using the 2.40 mm lens. The sample unit was a MS 1-Small Volume Sample Dispersion Unit stirred cell. The dispersant was DI water. The sample particle size distribution was assumed to follow a normal distribution.

Analysis model: polydisperse.

Setup presentation: standard wet (30HD)

Particle R.I.=(1.5295, 0.1)

Dispersant R.I.=1.33

### Procedure:

1 ml of Tween 20 was diluted to 1000 ml with water (solution 0.1% of Tween 20 in DI water). Approximately 250 mg of sample was dispersed in 20 ml of the solution 0.1% of Tween 20 in DI water. This sample was sonicated for 2 minutes and delivered dropwise to the previously filled and background corrected measuring cell until the desired obscuration was reached.
This dispersion (the dispersion in the stirring measuring cell) was measured after stabilization of the obscuration.
   v. X-ray Powder Diffraction (XRD)

The X-ray diffractograms were obtained using a RX SIEMENS D5000 diffractometer with a vertical goniometer and a copper anodic tube, radiation CuK_{α}, λ=1, 54056 Å.

### Example 1

### Preparation of an Enriched Mixture of Voriconazole Versus its (2S,3R)-enantiomer

To a mixture of racemic Voriconazole (60.6 g, 0.174 mol) in ethyl acetate (120 ml) (1S)-(+)-10-camphorsulfonic acid (40.3 g, 0.174 mol) and methanol (758 ml) were added. The mixture was heated to reflux temperature and approx. 270 ml of solvent were distilled under atmospheric pressure; in doing this, ethyl acetate was removed azeotropically. The resulting solution was cooled down to 20-24° C. and stirred for 1 hour and 45 minutes. The suspension thus formed was filtered without washings, obtaining a solid (33.94 g) which was dried at 50-60° C./vacuum (32.05 g.) The solid corresponds to (2S,3R)-enantiomer of Voriconazole (1S)-(+)-10-camphorsulfonate (enantiomeric purity: 98.76%, HPLC method A).

The mother liquor contains a mixture of Voriconazole (1S)-(+)-10-camphorsulfonate and (2S,3R)-enantiomer of Voriconazole (1S)-(+)-10-camphorsulfonate in an approx. ratio of 75/25 (HPLC method A).

Solvent from the mother liquors was distilled at atmospheric pressure until almost to dryness. Ethyl acetate (297 ml) was added and 105 ml of solvent were distilled at atmospheric pressure. The solution was cooled down to 20-25° C., aqueous saturated sodium bicarbonate solution (170 ml) was added slowly and the mixture was stirred for 10 minutes. The phases were allowed to settle and the organic layer was separated. The aqueous phase was re-extracted with ethyl acetate (159 ml). The combined organic phases were washed with water (21.2 ml), filtered and distilled under reduced pressure until almost to dryness to give a residue which correspond to a mixture of approx. 75/25 of Voriconazole and (2S,3R)-enantiomer of Voriconazole according to the analysis of the previous mother liquors (estimated content: 38.18 g).

### Example 2

### Preparation of Voriconazole (1R)-(−)-10-camphorsulfonate

To the residue obtained in example 1 (1R)-(−)-10-camphorsulfonic acid (25.39 g, 0.109 mol) and methanol (401 ml) were added. The mixture was heated to reflux temperature and 47 ml of solvent were distilled under atmospheric pressure. The resulting solution was cooled down to 5-8° C. and stirred for 2 hours and 30 minutes. The suspension thus formed was filtered to yield a wet white solid (37.91 g) which was dried at 50-60° C./vacuum to give Voriconazole (1R)-(−)-10-camphorsulfonate (36.13 g, 75.78% yield on the desired enantiomer).

Analytical data: HPLC enantiomeric purity (HPLC, method A): 99.46%, Chemical purity (HPLC, method B): 99.87%.

### Example 3

### Preparation of Voriconazole (1R)-(−)-10-camphorsulfonate

To a residue similarly obtained as in example 1 (6 g, 0.017 mol, mixture of approx. 63/37 of Voriconazole and (2S,3R)-enantiomer of Voriconazole) (1R)-(−)-10-camphorsulfonic acid (3.99 g, 0.017 mol) and methanol (55.5 ml) were added. The mixture was heated until complete solution, cooled down to 0-2° C. and stirred for 2 hours. The suspension thus formed was filtered to yield a wet white solid (5.40 g) which was dried at 50-60° C./vacuum to give Voriconazole (1R)-(−)-10-camphorsulfonate (5.16 g, 81.52% yield on the desired enantiomer).

Analytical data: HPLC enantiomeric purity (HPLC, method A): 99.24%, Chemical purity (HPLC, method C): 99.81%.

### Example 4

### Preparation of Voriconazole

The product obtained in Example 2 (Voriconazole (1R)-(−)-10-camphorsulfonate, 36 g, 0.062 mol) was suspended in ethyl acetate (149 ml) and aqueous saturated sodium bicarbonate solution (119 ml) was added slowly. The mixture was stirred for 10 minutes, the phases were allowed to settle and the organic layer was separated. The aqueous layer was re-extracted with ethyl acetate (112 ml). The combined organic layers were washed with deionised water (15 ml) and concentrated under vacuum until almost to dryness. Isopropanol (29.8 ml) was added and concentrated again under vacuum until almost to dryness. Isopropanol (67 ml) was added and the suspension was heated until complete solution (72° C.). The solution was cooled down to 0-2° C. and stirred for 1 hour and 15 minutes. The suspension formed was filtered, the cake was washed with cold isopropanol (7.5 ml). A white crystalline solid was obtained after drying at 50-60° C. under vacuum until constant weight: 19.56 g (90.47% yield).

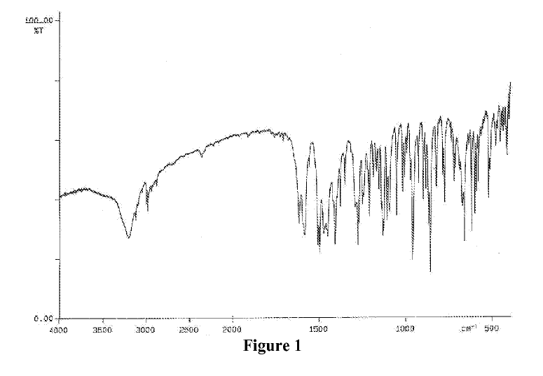

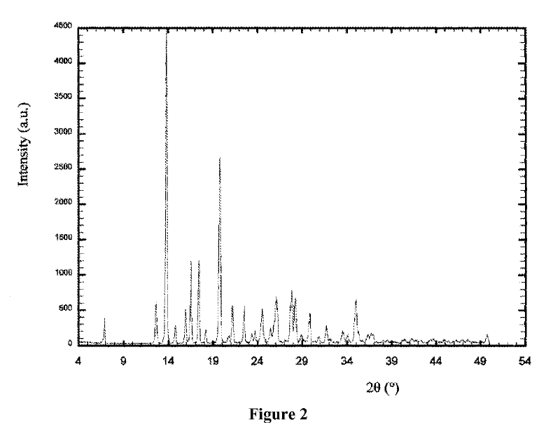

Analytical data: HPLC enantiomeric purity (HPLC, method A): 99.98%, Chemical purity (HPLC, method B): 99.96%, Assay (HClO₄): 100.56%, Loss on drying: 0.0%, Water content: 0.16%, IR: See FIG. 1, XRD (20): see FIG. 2, Particle Size Distribution: D(v, 0.1)=20.0 μm, D(v, 0.5)=50.9 μm, D(v, 0.9)=89.8 μm.

Having thus described in detail various embodiments of the present invention, it is to be understood that the invention defined by the above paragraphs is not to be limited to particular details set forth in the above description as many apparent variations thereof are possible without departing from the spirit or scope of the present invention.

What is claimed is:

1. A process for the preparation of Voriconazole (Compound I) 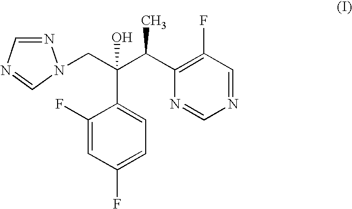
which comprises:
a) treating racemic Voriconazole (compound II) with (1S)-(+)-10-camphorsulfonic acid in an organic solvent to form a suspension containing (2S,3R)-enantiomer of Voriconazole (1S)-(+)-10-camphorsulfonate (compound IV); 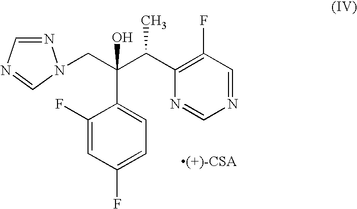
b) filtering the suspension and separating out the solid (2S,3R)-enantiomer of Voriconazole (1S)-(+)-10-camphorsulfonate (compound IV) to form a mother liquor which is enriched with Voriconazole (1S)-(+)-10-camphorsulfonate (compound V); 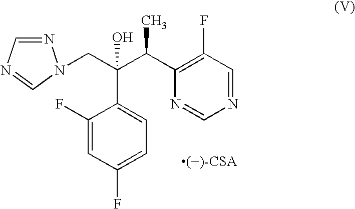
c) treating the mother liquor with with an organic solvent and an aqueous alkaline solution to obtain an enriched mixture of Voriconazole (compound I);
d) treating the enriched mixture of Voriconazole with (1R)-(−)-10-camphorsulfonic acid in an organic solvent to obtain Voriconazole (1R)-(−)-10-camphorsulfonate (compound III); 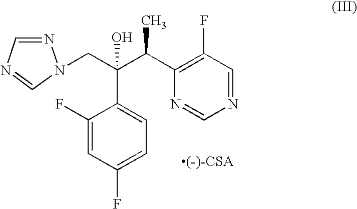
e) treating Voriconazole (1R)-(−)-10-camphorsulfonate (compound III) with an organic solvent and an aqueous alkaline solution to obtain Voriconazole (compound I); and
f) optionally purifying the Voriconazole.

2. Voriconazole with an enantiomeric purity of at least about 97.00% and a chemical purity of at least about 99.50%.

3. Voriconazole with an enantiomeric purity selected from the group consisting of about 97.50% to about 100% and a chemical purity selected from the group consisting of about 99.50% to about 100.00%.

4. The process of claim 1, wherein the mother liquor which is enriched with Voriconazole (1S)-(+)-10-camphorsulfonate has a ratio of Voriconazole (1S)-(+)-10-camphorsulfonate: (2S,3R) Voriconazole (1S)-(+)-10-camphorsulfonate of about 60: about 40 by weight to about 90: about 10 by weight.

5. The process of claim 4, wherein the mother liquor which is enriched with Voriconazole (1S)-(+)-10-camphorsulfonate has a ratio of Voriconazole (1S)-(+)-10-camphorsulfonate: (2S,3R) Voriconazole (1S)-(+)-10-camphorsulfonate of about 70: about 30 by weight to about 80: about 20 by weight.

6. The process of claim 5, wherein the mother liquor which is enriched with Voriconazole (1S)-(+)-10-camphorsulfonate has a ratio of Voriconazole (1S)-(+)-10-camphorsulfonate: (2S,3R) Voriconazole (1S)-(+)-10-camphorsulfonate of about 75: about 25.

7. The process of claim 1, wherein the mother liquor treated with a base and enriched with Voriconazole has a ratio of Voriconazole: (2S,3R) Voriconazole of about 60: about 40 by weight to about 90: about 10 by weight.

8. The process of claim 7, wherein the mother liquor treated with a base and enriched with Voriconazole has a ratio of Voriconazole: (2S,3R) Voriconazole of about 70: about 30 by weight to about 80: about 20 by weight.

9. The process of claim 8, wherein the mother liquor treated with a base and enriched with Voriconazole has a ratio of Voriconazole: (2S,3R) Voriconazole of about 75: about 25.

10. The process of claim 1, wherein the organic solvent is selected from the group consisting of alcohols, esters, ketones, ethers, nitrites, hydrocarbons and mixtures thereof.

11. The process of claim 10, wherein the organic solvent is selected from the group consisting of C₁-C₄ alcohols, methyl acetate, ethyl acetate and mixtures thereof.

12. The process of claim 11, wherein the organic solvent is selected from the group consisting of methanol, isopropanol, ethyl acetate and mixtures thereof.

13. The process of claim 12, wherein the organic solvent of steps a) and d) is methanol.

14. The process of claim 12, wherein the organic solvent of steps c) and e) is ethyl acetate.

15. The process of claim 1, wherein the aqueous alkaline solution is a saturated sodium bicarbonate solution.
